(19) United States
(12) Patent Application Publication
(10) Pub. No.: US 2026/0232278 A1
(43) Pub. Date: Aug. 13, 2026

(54) AI-BASED HEALTH RISK TRACKERS

(71) Applicants: Robert Fisher, Lakewood Ranch, FL (US); Richard Fessler, Lakewood Ranch, FL (US)

(72) Inventors: Robert Fisher, Lakewood Ranch, FL (US); Richard Fessler, Lakewood Ranch, FL (US)

(73) Assignee: Health Magnet Holdings, Lakewood Ranch, FL (US)

(21) Appl. No.: 19/454,834

(22) Filed: Jan. 21, 2026

Related U.S. Application Data
(60) Provisional application No. 63/757,249, filed on Feb. 11, 2025.
(60) Provisional application No. 63/757,080, filed on Feb. 11, 2025.

(51) Int. Cl. A61B 5/00; A61B 5/0205; A61B 5/022; A61B 5/024; A61B 5/1455; G16H 40/67; G16H 50/30

(52) U.S. Cl. CPC A61B 5/7275; A61B 5/0205; A61B 5/14551; A61B 5/742; A61B 5/746; G16H 40/67; G16H 50/30; A61B 5/022; A61B 5/02405; A61B 5/681

(57) ABSTRACT

The present disclosure relates to a system for continuous disease risk monitoring. The system includes an input device to continuously collect health-related parameters of a patient in real time. Further, a cloud-based server stores data of the health-related parameters in a database and a processing module establishes patient specific baselines for the health-related parameters of the patient. Upon comparing current values of the health-related parameters to the patient specific baselines, a daily data trendline is generated. Based on the daily data trendline, at least one of congestive heart failure (CHF) decompensation, secondary stroke risk, and vascular health risk may be identified and patient's health status can be classified into risk levels thereof. Thereupon, based on the classified risk levels in the daily data trendline, visual risk alerts are trigger. The disclosed system offers a monitoring platform for classifying a patient's health risk status depending upon the severity.

### FIELD OF THE INVENTION

Embodiments of the present invention relate to the field of software-based health analytics, in general and specifically relates to providing remote monitoring solutions to analyze risks in patients prone to cardiovascular diseases.

### DESCRIPTION OF THE RELATED ART

Wearable devices, such as smartwatches and fitness trackers, have become widely adopted and enable continuous monitoring of users' health-related parameters. In addition, sensor-based health monitoring devices deployed in home environments facilitate the collection of health-related parameters, including step count, heart rate variability (HRV), blood pressure, sleep metrics, and stress indicators, often in real time.

Modern data analytics, combined with recent advances in wearables and remote patient monitoring (RPM) solutions, provide enormous quantities of real-time biometric data. However, many existing solutions simply display raw metrics, which alone can overwhelm users.

Individuals affected by cardiovascular diseases, who are at elevated risk of relapse events including heart failure and stroke, frequently self-monitor their health-related parameters on a regular basis. However, such monitoring is not consistently assessed or acted upon to prevent high risk or severe events.

By way of example, patients with congestive heart failure (CHF) or significant cardiovascular risk often experience subtle physiological or behavioral changes, such as rising heart rates, decreasing oxygen saturation (SpO₂), and reduced activity, for days or even weeks before an acute exacerbation. However, traditional clinical workflows rely on periodic checks of vital parameters, intermittent patient monitoring, and patient self-reporting, making it challenging to detect early warning signs before they escalate into emergency department visits or hospital admissions.

By way of another example, stroke survivors often face a heightened risk of secondary stroke, particularly when uncontrolled hypertension persists. Traditional approaches to blood pressure (BP) monitoring, such as intermittent checks in a clinical setting or periodic home measurements, may not capture frequent spikes in systolic BP that could indicate a potentially worsening condition.

Thus, effective conversion of raw biometric data into actionable health insights remains a significant technical challenge in tracking high-risk conditions in patients with cardiovascular diseases.

### SUMMARY OF THE INVENTION

Embodiments of the present invention may relate to a system for continuous disease risk monitoring. The system may include an input device, a cloud-based server, a processing module, and a dynamic interface. The input device may continuously collect health-related parameters of a patient in real time. Further, the cloud-based server may store data of the health-related parameters in a database. The processing module may then establish patient specific baselines for the health-related parameters of the patient. Further, the processing module may compare current values of the health-related parameters to the patient specific baselines to generate a daily data trendline. Based on the daily data trendline, the processing module may identify at least one of congestive heart failure (CHF) decompensation risk, secondary stroke risk, and vascular health risk and classify patient's health status into risk levels thereof. Thereafter, the processing module may trigger visual risk alerts, based on the classified risk levels in the daily data trendline. Finally, the dynamic interface communicatively connected to the processing module may send the visual risk alerts to the patient and clinicians. The visual risk alerts reflect the severity of CHF decompensation, secondary stroke, or vascular health. By visually alerting clinicians or healthcare teams to subtle deteriorations, the disclosed invention allows earlier interventions, such as medication adjustments or telehealth visits which ultimately reduces hospital admissions.

In accordance with an embodiment of the present invention, the input device may be one of a wearable device, digit-based BP device or standard BP cuff, patch, and connected electrocardiogram (ECG) band or patch, and equivalents thereof. The disclosed invention uses the wearable devices or sensor-based health monitoring devices to collect the data pertaining to the health-based parameters which facilitates in continuously tracking health-related parameters and respond quickly when patients show early signs of cardiovascular diseases through abnormal readings.

In accordance with an embodiment of the present invention, the processing module may include at least one of an AI-based module or a ML based module. By monitoring different aspects of health like vascular, systolic, activity and sleep, through the health-related parameters, the AI modules help in identifying risk levels associated with different diseases.

Additionally, embodiments of the present invention may also include a method for continuous monitoring of cardiovascular diseases, implemented by the processing module. The method may include continuously collecting health-related parameters of a patient, in real time, by a plurality of input devices. The method may also include storing data of the health-related parameters in a database, establishing patient specific baselines based on the health-related parameters of the patient, and comparing current values of the health-related parameters to the patient specific baselines to generate a daily data trendline. Further, based on the daily data trendline, the method recites identifying risk of a cardiovascular disease and classifying the patient's health status into risk levels thereof. Also, based on classified risk levels in the daily data trendline, the method includes triggering visual risk alerts. The visual risk alerts reflect the severity of CHF decompensation, secondary stroke, or vascular health like diseases thereby alerting clinicians to subtle deteriorations in the health status of the patient, for faster interventions and reducing hospital admissions.

In accordance with an embodiment of the present invention, the health-related parameters may include biometric parameters of the patient. The biometric parameters may be at least one of heart rate (HR), heart rate variability (HRV), oxygen saturation (SpO2), step/activity data, and blood pressure of the patient, which may be collected on hourly or daily basis.

In accordance with an embodiment of the present invention, the data may be collected on hourly or daily basis. Thus, continuous real-time collection, on hourly or daily basis, of the plurality of the biometric parameters from the input devices, such as wearables or sensor-based monitoring devices can predict worsening heart failure status and secondary stroke status.

In accordance with an embodiment of the present invention, a composite risk score may be generated by analyzing both absolute deviations and frequency of change of the health-related parameters from the collected data. Thus, the composite risk scores enable timely clinical intervention to prevent critical health situations.

In accordance with an embodiment of the present invention, the plurality of health-related parameters may be collected in one of every 15 minutes, 30 minutes, within a pre-defined time of five days. By automatically capturing the health-related parameters, like systolic BP at four intervals per hour, an optimum average value can be calculated.

In accordance with an embodiment of the present invention, the method may include summing weighed data obtained from the health-related parameters on an hourly basis. Further, the method recites calculating a daily average score from the summation of the weighed data of the health-related parameters. Based on the daily average score, the method includes classifying the patient's health status into one of three tiers from one of good health, risk, or elevated risk.

In accordance with an embodiment of the present invention, the method may include averaging hourly totals of the data of the health-related parameters to generate a daily risk score, after 24 hours.

In accordance with an embodiment of the present invention, the method may include establishing new baselines and threshold levels as the patient's health related parameters improve or decline over time. Establishing new baselines and threshold levels as the patient's health related parameters change, enables a seamless risk assessment.

In accordance with an embodiment of the present invention, the method may include continuously refining alert thresholds based on one or more validated patient outcomes of the patient's health status.

In accordance with an embodiment of the present invention, the method may include sending automated alerts via SMS, email, or EHR notifications to one of the patient and clinicians, via a dynamic interface communicatively connected to the processing module, when the patient's health status transitions to an elevated risk level. Thus, continuous real-time collection, on hourly or daily basis, of the plurality of the biometric parameters from the input devices, such as wearables or sensor-based monitoring devices can predict worsening heart failure status and secondary stroke status. by providing a simple and actionable interface to clinicians.

In accordance with an embodiment of the present invention, the cardiovascular disease can be identified as at least one of congestive heart failure (CHF) decompensation, secondary stroke risk, and vascular health risk to the patient. Thus, the risk levels in major cardiovascular diseases can be monitored for preventing exacerbations.

In accordance with an embodiment of the present invention, the method may include tracking hear rate Variability (HRV) and downward data trendline of SpO2 levels for multi-days. By capturing HRV and multi-day downward trends, clinicians may intervene before patients experience severe CHF exacerbations.

In accordance with an embodiment of the present invention, the daily data trendline may include one of mild, moderate, and severe levels corresponding to early warning, caution, and high alert levels, respectively, based on both absolute values and deviations from the patient specific baseline. By way of presentation of the risk levels as warning, caution, and high alert levels, severity of the disease is reflected by which a clinician may easily differentiate normal situation from an emergency situation.

Further, embodiments of the present invention may also include a method for monitoring blood pressure (BP) to predict secondary stroke risk implemented by a processor in an AI module. The method may include sampling systolic BP through an input device, at pre-determined intervals in a pre-defined time. Further, the method may include sending the samples of the systolic BP to a central server, computing a daily mean from the samples of the systolic BP, and tracking multi-day data trendline from the daily mean and classifying the patient's health status into risk levels. Also, based on the risk levels, the method may include triggering visual risk alerts. Through the disclosed method, potential progression of hypertension or imminent risk for secondary stroke may be indicated.

In accordance with an embodiment of the present invention, the pre-determined intervals may be one of 15 minutes or 30 minutes, and the pre-defined time may be five days.

In accordance with an embodiment of the present invention, for sampling the systolic BP, the input device may be either a digit-based BP device or a standard BP cuff. Such devices enable continuous monitoring thereby providing a quick overview of the patient's health.

In accordance with an embodiment of the present invention, for tracking the multi-day data trendline, the daily means of the samples of the systolic BP may be analyzed for five-days via machine learning (ML) or statistical methods.

In accordance with an embodiment of the present invention, the method may include excluding an oldest data point when each new day's mean enters a five-day window.

In accordance with an embodiment of the present invention, the method may include providing trend graphs and the visual risk indicators. Also, the method may also include providing automated alerts as one of an SMS, email, and electronic health record (EHR) notifications. Herein the above steps are implemented via a dynamic interface communicatively connected to the processing, when the patient's health status transitions to an elevated risk level.

In accordance with an embodiment of the present invention, the method may include bypassing the daily mean computation steps when a single systolic BP reading spikes above a critical threshold and sending urgent warnings to clinicians.

Additionally, embodiments of the present invention may also include a method for indicating vascular health risk, implemented by a processor in an AI module. The method may include continuously collecting data of plurality of health-related parameters of a patient, from a plurality of input devices. Further, the data from the plurality of health-related parameters may be stored in a database. The method may then include assigning weighted points for each health-related parameter. Thereafter, the method includes summing weighed data obtained from the health-related parameters, on an hourly basis, and calculating a daily average score thereof. Thereupon, based on the daily average score, the method may include classifying the patient's health status into risk levels and triggering the risk levels as visual risk alerts. As a result, a seamless assessment of risk level of the vascular health is possible without any extra burden on capabilities of the existing wearables.

In accordance with an embodiment of the present invention, the method may include ingesting data from a plurality of input devices from one of smart rings, smartwatches, blood pressure cuffs, and other combinations of wearable devices.

In accordance with an embodiment of the present invention, the method may include averaging the hourly totals, after every 24 hours, to generate a daily average score.

In accordance with an embodiment of the present invention, the patient's health status may be classified into risk levels as good health, risk, or elevated risk. Upon detecting the patient's health status trending into the elevated risk level, the method may include triggering immediate alerts to health care teams via a dynamic interface communicatively connected to the processor. Thus, an intuitive interface can display real-time alerts by checking hourly metrics.

In accordance with an embodiment of the present invention, the method may include examining historical data of the patient, with a history of congestive heart failure (CHF) or comorbidities, for patterns or anomalies for analyzing impending health status. Accordingly, the present method provides actionable and predictive insights.

In accordance with an embodiment of the present invention, the method may include implementing feedback steps from patient outcomes with one of successful interventions by clinicians and reduced hospital readmissions.

In accordance with an embodiment of the present invention, the method may include triggering immediate notifications as SMS, email, or alerts to clinicians, via a dynamic interface communicatively connected to the processor, when the patient's health status transitions to an elevated risk level. Thus, a swift intervention is possible by clinicians in case of elevated risk level.

The invention could also extend to other areas of healthcare or other fields where there is a demand for analytics, finding trends, and comprehensive insights, among others.

### BRIEF DESCRIPTION OF THE DRAWINGS

So that the manner in which the above-recited features of the present invention is understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

The invention herein will be better understood from the following description with reference to the drawings, in which:

FIG. 1 illustrates a block diagram of a system for continuous disease risk monitoring, in accordance with an embodiment of the present invention;

FIG. 2 illustrates a flowchart for continuous monitoring of cardiovascular diseases, in accordance with an embodiment of the present invention;

FIG. 3 illustrates a flowchart further illustrating the method from FIG. 2, in accordance with one or more embodiments of the present disclosure.

FIG. 4 illustrates a flowchart further illustrating the method from FIG. 2, in accordance with an embodiment of the present invention; and

FIG. 5 illustrates a flowchart of a method for monitoring blood pressure to predict secondary stroke risk, in accordance with an embodiment of the present invention;

FIG. 6 illustrates a flowchart of a method for indicating vascular health risk, in accordance with an embodiment of the present invention; and

FIG. 7 illustrates an exemplary view of an interface depicting AI-driven health risk indicators, in accordance with an embodiment of the present invention.

It should be noted that the accompanying figures are intended to present illustrations of exemplary embodiments of the present disclosure. These figures are not intended to limit the scope of the present disclosure. It should also be noted that the accompanying figures are not necessarily drawn to scale.

### DETAILED DESCRIPTION OF THE INVENTION

In the following detailed description, numerous specific details are set forth in order to provide a thorough understanding of the embodiment of the invention as illustrative or exemplary embodiments of the invention, specific embodiments in which the invention may be practiced are described in sufficient detail to enable those skilled in the art to practice the disclosed embodiments. However, it will be obvious to a person skilled in the art that the embodiments of the invention may be practiced with or without these specific details. In other instances, well-known methods, procedures and components have not been described in detail so as not to unnecessarily obscure aspects of the embodiments of the invention.

The following detailed description is, therefore, not to be taken in a limiting sense, and the scope of the present invention is defined by the appended claims and equivalents thereof. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. References within the specification to “one embodiment,” “an embodiment,” “embodiments,” or “one or more embodiments” are intended to indicate that a particular feature, structure, or characteristic described in connection with the embodiment is included in at least one embodiment of the present invention.

The terminology employed in the present disclosure is utilized to delineate specific embodiments and does not aim to restrict the scope of the invention. In this context, the term “and/or” encompasses all possible combinations of one or more items listed in association. Further, the terms “a” and “an” herein do not denote a limitation of quantity, but rather denote the presence of at least one of the referenced items.

The conditional language used herein, such as, among others, “can,” “may,” “might,” “may,” “e.g.,” and the like, unless specifically stated otherwise, or otherwise understood within the context as used, is generally intended to convey that certain embodiments include, while other embodiments do not include, certain features, elements and/or steps.

Unless explicitly defined otherwise, all terms, including technical, technological, engineering, and scientific terminology, utilized in this document are presumed to carry the same connotations as commonly understood by individuals possessing ordinary skill in the pertinent field to which this invention pertains. Moreover, it is emphasized that terms, including those cataloged in commonly referenced dictionaries, should be construed to align with their intended meaning within the context of the relevant art and the disclosures provided herein. Any interpretations of terms should refrain from adopting an excessively formal or idealized stance unless explicitly delineated within the present disclosure.

When discussing the invention, it is important to recognize that various techniques and steps are disclosed, each offering distinct advantages and capable of being employed independently or in combination with one another. Therefore, this description avoids redundant enumeration of all possible combinations of individual steps to maintain clarity. However, it should be noted that such combinations are fully encompassed within the scope of the invention and the accompanying claims. Consequently, the specification and claims should be interpreted with the understanding that these combinations are permissible and fall within the ambit of the invention.

When perceiving the arrows between the components, it is understood that the direction of the arrow is only to depict the flow of the request and response highlighting the upstream and downstream systems and it does not restrict or omit the possibility of data that can flow in either direction of the arrow. When there is no arrow connecting any two or more components it only addresses an optimized path flow to achieve the desired goals and meet the needs and it does not restrict or omit the possibility of connectivity required to address any alternate flows the system needs to address for the optimal function.

As used herein, a ‘module’ which can be configured to apply trained AI models may be implemented in hardware, software, firmware, or any combination thereof, and may include rule-based logic, machine-learning logic, or hybrid approaches.

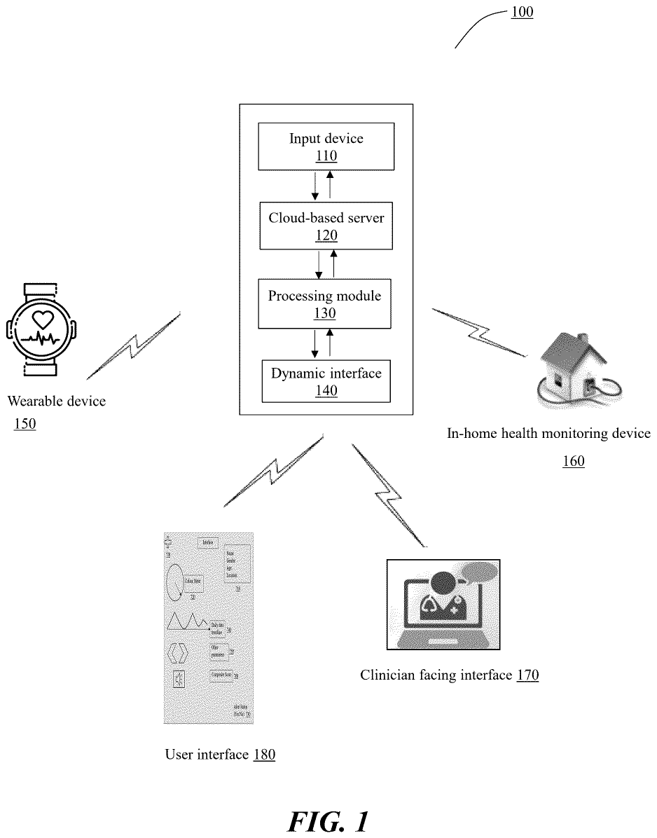

FIG. 1 illustrates a block diagram that describes a system 100 for continuous disease risk monitoring, in accordance with an embodiment of the present invention. The system 100 may include an input device 110, a cloud-based server 120 (hereinafter referred to as ‘server’), a processing module 130, and a dynamic interface 140. Further, the system 100 may be connected to wearables 1, 2, . . . n (other wearables may be connected with the system and are not shown in the FIG. 1 for the sake of brevity) 160. It is also to be noted that depending upon the requirement of users or patients (if suffering from diseases-conditions), any number of wearables 150 could be connected to the system 100.

The input device may 110 continuously collects health-related parameters of a patient in real time. The input device 110 may be one of a wearable device 150 (hereinafter referred to as ‘wearables’).

In an example, the wearables 150 may be one of a fitness tracker, smartwatch, a smart bracelet, a smart belt, and a smart ring remotely connected to the cloud-based server 120 among others.

In an exemplary embodiment, the wearables 160 may include more advanced devices like, digit-based BP device or standard BP cuff, patch, and connected electrocardiography (ECG) band or patch, and equivalents thereof which render precise tracking of cardiovascular health and other critical parameters.

In an exemplary embodiment, the data pertaining to health-related data may be collected by the wearable 150 worn by a user or sensor-based health monitoring devices 160 (or in-home health monitoring devices). The health-related data may relate to a plurality of biometric parameters and may be one of the following: heart rate (HR), heart rate variability (HRV), systolic blood pressure (BP), blood oxygen saturation (SpO₂), steps or activity, sleep metrics, and stress scores, among others, biometric data.

The wearables 150 may include a plurality of sensors to enable such measurements in the user's body. The server 120 and wearables 150 are connected to the dynamic interface 140 and together forms an interconnected health monitoring platform (or AI-powered monitoring platform).

The sensor-based health monitoring devices 160 (or in-home health monitoring devices) may include glucose monitoring devices, blood pressure measuring devices, pulse oximeters, smart thermometers, smart inhalers, connected weighing scales, environmental sensors, motion sensors, and smart pill dispensers, among others connected to the server 120.

It is to be noted that the wearables 160 may rely on internet of things (IoT) connectivity to share the biometric data to a clinician through the cloud-based servers 110, ensuring prompt responses or intervention in case of high-risk levels of disease conditions. For example, narrowband IoT (NB-IoT) which is a cellular technology for low-power devices communicating over long distances could be used by the wearables.

The wearables 150 and the in-home health monitoring devices 160 (combinedly referred to as ‘tracking devices’) also empower users by offering a clear view of their bio metrics, promoting better self-management. These tracking devices provide reliability, accuracy, and seamless integration into the interconnected health monitoring platforms.

In an example, the wearables 150 may include heart rate monitors, accelerometers, and gyroscopes, along with temperature sensors. Through measuring the patient's pulse, heart rate monitors provide insights into cardiovascular health. Further, the accelerometers track movement and physical activity levels, while gyroscopes help in determination of orientation and rotation, and the combination is used for motion detection. By checking the body temperature, temperature sensors contribute to overall health assessments. These sensors collectively enable the wearables 160 to analyze the biometric data effectively, providing the patients and the clinicians with valuable data for remote therapeutic management.

The sensors along with pre-stored algorithms monitor the biometric data in real-time. For instance, heart rate monitoring is achieved through photoplethysmography, which measures blood flow changes. Additionally, wearables 150 can measure blood oxygen levels using pulse oximetry technology, thus providing insights into respiratory health.

In an exemplary embodiment, stress levels of the patient may be monitored to classify risk levels of diseases. For monitoring the stress levels, stress scores or indexes may be measured by the wearable 150 to offer a proprietary stress index via HRV or electrodermal activity (EDA). In an example, a stress score above 150 can indicate excess stress. It is to be noted that a resting heart rate (RHR) counts how many times a heart beats in a minute when one is at rest, whereas the HRV measures the variation in time between each heartbeat metric. The tiny fluctuations between the heartbeat metric are measured in milliseconds and are known as R-R intervals. In an example, if a patient's heart beats 60 times in a minute, those beats may not be necessarily spaced evenly. One interval might be 980 ms, and the next may be 1030 ms. That difference between the two beats refers to as the heart rate variability or HRV. The higher the HRV, the more flexible and resilient the body of a patient is and the body can smoothly shift between stress and recovery, in contrast, a low HRV means the stress response of the patient's body is never turned off and the body stays on alert, even when one is trying to rest.

The wearable 150 may be calibrated to measure the above listed biometric parameters so that there is an ignorable difference between lab results in clinic and that of the values shared by the wearable.

In an aspect, the value of the data collected from the wearables 150 could be shared by an application installed in a digital device like smartphone or a tablet etc.

In another aspect, the values of the data may directly be shared with the server 110 through the wearable 150. In such a case, an application shall be installed in the wearable 150 for establishing communication with the server.

The server 110 receives the data pertaining to a plurality of biometric parameters (shortened as ‘biometric data’) from the wearable 150 and from the in-home health monitoring device 160) and store the received data either into a single datastore categorized for storing multiple types of information or separate datastore (the datastores are not shown in the figures). The datastore may store the health-related data of the patient for retrieval and processing. In the datastore, the health-related data of the patient may be stored in variety of forms. In an example, the health-related data of the patient may be stored in a structured or unstructured or semi-structured form.

Further, to ensure data privacy and security, sensitive health-related data of the patient may be anonymized in the datastore. Encryption protocols may be employed to safeguard both the data and its representations during storage and transmission.

In an example, the health-related data of the patient could be stored as graph or hybrid of multiple forms. In another example, the health-related data of the patient may be stored in any other form or format for an efficient retrieval. Herein, patient related data refers to the biometric data and disease conditions, for example, CHF and stroke. The biometric data is then processed to calculate a composite score (CS) by a composite scoring engine. Herein, the composite score (CS) reflects the user's health risk level. Also, the composite scoring engine may parallelly process multiple types of data inputs (ex: biometric data) thereby speeding up the overall inference process of risk analysis.

In accordance with an embodiment, the composite scoring engine may refer to an AI or ML based engine to generate the composite score (CS). The AI or ML based engine may apply smoothing/rolling averages to the historical trends or graphs so as to avoid rapid toggling between states. The AI or ML based engine may act as a dedicated composite scoring engine to parallelly process multiple types of data inputs (biometric data and other parameters related data) thereby speeding up the overall inference process.

The composite scoring engine may calculate timed updates to an individual's health risk level based on the received data streams (biometric data) mapped with respect to individualized baselines (the CS may be compared with a threshold CS value and based on the comparison a risk status may be generated) and other inputs from users, for example, previous history of comorbidities, such as diabetes and BP, etc. it is to be noted that point assignments may be modified to fit specific patient populations, comorbidities, or care protocols.

In accordance with an embodiment, a rules-based alert module may issue alert to the clinicians when threshold values corresponding to the calculated health risk level of the plurality of biometric parameters may be exceeded. In an example, the system 100 may also support unique clinical protocols (e.g., different thresholds for advanced CHF patients) and scales across large patient populations.

The server 110 may receive continuous health-related data from a user's body. Further, the server 120 may store data of the health-related parameters in a database. The server 110 may be configured to receive health-related data for a plurality of users. Further, the server 110 may store and process the health-related data for creating patient specific baselines for each user of the plurality of users.

The cloud-based server or the server 120 helps in centralizing and storing the heath-related data for a comprehensive analysis and accessibility across various platforms (e.g. mobile phone, laptop). By way of the storage of the user health-related data at the servers 120, the clinicians can access the data at anytime, anywhere, for continuous clinical surveillance better based and swift intervention. The user health-related data may be stored as individual electronic health records (EHRs) in libraries with complete information of course of treatment. The EHRs can then be analyzed using AI modules to identify daily trends, predict potential health risks, and personalize treatment plans. Moreover, advanced analytics tools, integrated within the server may allow for predictive modelling for chronic disease management thereby reducing hospital readmissions.

The disclosed system 100 may offer end-to-end encryption, role-based access control, audit trails for data review thereby complying with standard regulatory frameworks, such as the health insurance portability and accountability act (HIPAA) in the U.S. The present system 100 may also support anonymized or pseudonymized data usage for AI model training wherever mandated by regional laws considering the general data protection regulation (GDPR) in Europe. Such regulations regulate that collection, storage, and processing of the health-related data of the patients must comply with the protection of individual privacy. Moreover, the present cloud-based architecture is designed to handle large patient populations and 24/7 data ingestion.

The server 120 may receive data streams from the plurality of wearables 150 or in-home health monitoring device 160 connected via a remote connection and store the patient specific baselines generated from the data streams.

In accordance with an embodiment of the present invention, for generating the patient specific baselines from the data pertaining to the health-related parameters, a pre-defined period is considered which may vary from at least two weeks to a maximum limit set by the clinician. The span for monitoring a particular user is set by the clinician for getting optimized analysis. It is to be noted that a baseline is calculated for each parameter of the health-related parameters, forming the foundation against which future deviations in the health-related parameters are analyzed.

In an implementation of the present invention, for generating baselines, data relating to resting HR ranges, typical SpO₂ levels, and average daily steps may be collected. The system may monitor and generate a daily data trendline for reflecting change in baseline resting HR and may also track variations in beat-to-beat intervals (HR Variability). The “daily data trendline” may be defined as a recurring or observable behavior of the analyzed data (ex: pertaining to the biometric data) over a day. Similarly, rather than focusing on single dips, the disclosed system observes multi-day downward trends which could signal respiratory or circulatory stress to the patient. It is to be noted here the term ‘multi-day’ refers to extending over consecutive or inconsecutive days. Also, for monitoring patients with CHF risk, a consistent decline in step count over consecutive days may indicate reduced exercise tolerance and early heart failure progression with respect to periodic or context-based measurements (day vs. night activity) tracking.

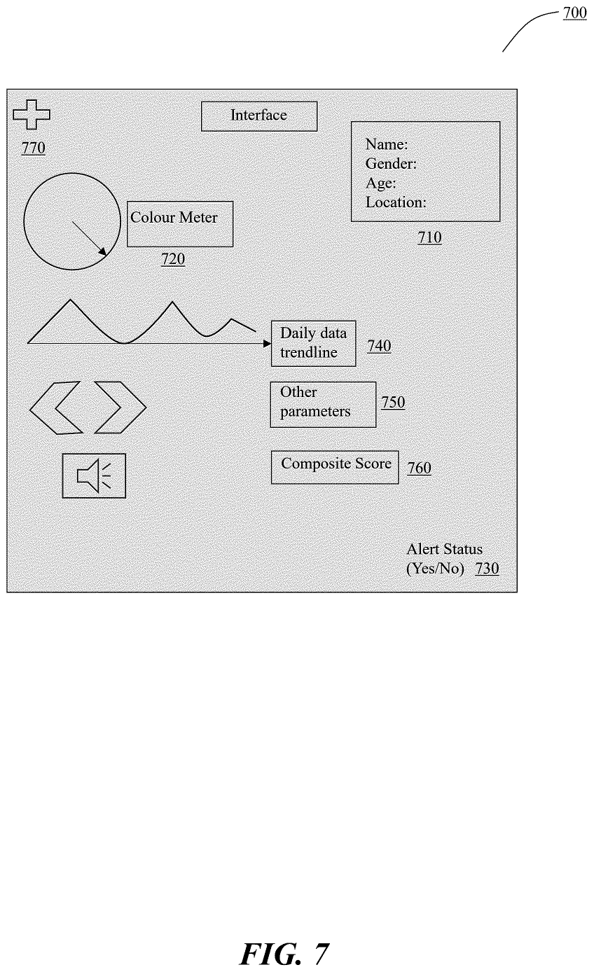

The server 110 can be accessed by the clinician via a dynamic interface (explained in below FIG. 7) which is part of an application dashboard on a display device for checking the composite score. In an example, the clinician may be a healthcare professional.

The processing module 130 may be at least one of an AI-based module or a ML based module. The processing module 130 establishes patient specific baselines for the health-related parameters of the patient. Further, the processing module 130 compares current values of the health-related parameters to the patient specific baselines to generate a daily data trendline. Thereupon, based on the daily data trendline, the processing module 130 may identify at least one of congestive heart failure (CHF) decompensation, secondary stroke risk, and vascular health risk and classifies patient's health status into risk levels of these diseases.

The AI-based module may include a modular AI-based engine (not shown in Figures) which may host a plurality of disease-specific AI modules for analyzing different aspects of health on the collected data pertaining to the health-related parameters using the individualized baselines. In an example, by monitoring vascular indicators (e.g., near-systolic and near-diastolic measurements) stroke risk and congestive heart failure (CHF) can be predicted. Similarly, by monitoring systolic blood pressure trend analysis, secondary stroke risk predictions can be done, by means of the plurality of disease-specific AI modules.

In an embodiment, the modular AI-based engine may include the composite scoring engine and the rules-based alert module.

Further, the modular AI-based engine may includes at least one large language model (LLMs) configured to generate text-based clinical summaries of data trends of the user's physiological measurements. The AI-driven summarization of large data sets for quick interpretation (e.g., ChatGPT-like LLM integration) automates the process of taking clinician notes. Further, compatibility of the AI enabled health analytics platform with RTM workflows enables near real-time monitoring, intervention, and billing (if applicable), while preserving clinician oversight and decision authority. The modular AI-based engine also refines threshold levels of the plurality of biometric parameters for disease conditions, baselines, risk assessment, and predictive models over time, leveraging population-level data or individual patient feedback to enhance accuracy and relevance.

In an example, fine-tuning and prompt-engineering techniques enable adaptation of these models to specific clinical contexts, patient populations, or disease categories without retraining entire model architectures. In addition, feedback derived from real clinical settings may be used to iteratively refine model behavior, address performance variability across specialties, and improve reliability in complex or rare disease conditions.

It is to be noted that the modular AI-based engine, the composite scoring engine, and the rules-based alert module may include custom algorithms, logic functions, executed by GPUs or microcontroller(s), such as ARM processors, x86 processors, DSPs or ASICs. Particularly, DSPs are designed for real-time signal processing tasks such as audio, video, and telecommunications and used in speech recognition and audio processing software. ASICs may be custom-designed for specific software tasks and thus may be used in high-efficiency computing for niche applications.

The dynamic interface 150 may be communicatively connected to the processing module 130 to send the visual risk alerts to the patient and the clinicians via the application dashboard (shortened as “dashboard” hereinafter).

The application dashboard may include a clinician-facing web dashboard or interface 170 and a consumer or user or patient-facing interface (web or mobile) 180. The clinician-facing web interface 170 may deliver real-time alerts and visual risk alerts (via stoplight or numeric indicators), and patient management tools. Also, the clinician-facing web interface 170 may depict in-depth historical views, day-to-day or hour-to-hour comparisons, and correlation analytics (e.g., how changes in sleep correlate with BP fluctuations). In addition, patient roster may be depicted with filtering (e.g., show only those flagged as red) and integration with EHR systems is shown via standard APIs like health level seven international (HL7)'s fast healthcare interoperability resources (FHIR) which is a standard for exchanging health information electronically.

Further, through the consumer or user facing interface 180, end-users or patients can track personal health trends, receive notifications as personal health insights, and access educational resources. The user facing interface 180 may provide dynamic charts, easy-to-understand indicators, and self-care tips. Moreover, the user facing interface 180 may provide one of daily check-ins, medication reminders, alerts and nudges (e.g., prompts to increase daily steps if activity trends are declining).

Thus, as disclosed in the present invention, the disclosed system 100 can automatically identify when data indicate a worsening condition, prompting interventions, telehealth consultations, or medication adjustments under recognized RTM billing frameworks.

As used herein, the term “interface” refers to a functional system component implemented using one or more processors or processing module 130 and the cloud-based server 120 that enables interaction between the patient and the disclosed system 100. The interface is configured to receive inputs and to generate outputs based on computational processing performed using the processing module 130, by the system 100 which includes analysis of biometric data, determination of the patient's health status, or generation of risk levels. The interface 140 may be implemented using graphical, auditory, haptic, or other modalities and may be provided via one or more devices, such as a user equipment, for example, a mobile device, a tablet, and a laptop. The interface is not limited to the presentation of information, but operates in coordination with underlying processing logic to adapt outputs in response to changes in system state, detected disease conditions, or patient interactions.

The disclosed interface 140 or any other interfaces which have been discussed in the present invention may be designed using the coding languages such as, javascript, python, and SQL, etc. Further, various open-source frameworks such as Note JS, flutter, react native, Xamarin, iconic framework, codova, asp.net, and nativescript, etc. may be employed to create the frontend. The disclosed interface support multimodal interaction by enabling users to toggle between various input methods seamlessly. Additionally, the interface adapts to different screen sizes and resolutions, thereby ensuring optimal rendering of the output across various devices used by the patients and the clinicians. In an example, the wearables 150 and in-home health monitoring devices 160, equipped with the sensors, collect the biometric data and then use remote connection technologies to send the data to the server 110. The remote connection may be established primarily through a variety of wireless communication technologies.

In another example, the wearables 150 may be paired with the digital devices like smartphones or computers and then the collected data may be shared with the server 120.

In some embodiments, low-power wide-area network (LPWAN) technologies, such as LoRaWAN or Narrowband IoT (NB-IoT), may be employed to enable long-range communication with minimal power consumption. Such technologies are particularly suitable for battery-operated sensors in the wearables 160 and in-home monitoring devices deployed in environments with limited infrastructure or requiring deep indoor coverage. These communication protocols support continuous clinical surveillance by enabling periodic or event-driven transmission of health-related data over extended durations.

In certain embodiments, short-range wireless communication protocols, such as Bluetooth Low Energy (BLE), ZigBee, or Z-Wave, may be used to interconnect wearables 150, in-home health monitoring devices 160, with the server 120. For instance, BLE is commonly used for direct communication between wearables and digital devices, such as smartphones or tablets, for energy efficiency reasons thereby allowing continuous data transfer without significantly draining the wearable's battery. Due to the widespread adoption of Bluetooth in fitness trackers and smartwatches, this method of transmission is commonly validated which enables real-time monitoring and data synchronization with mobile applications. While ZigBee or Z-Wave may support mesh-based sensor networks within a residential or assisted-living environment. Such configurations allow physiological data, activity data, or environmental data to be collected from multiple devices and aggregated locally prior to transmission for continuous clinical surveillance.

In further exemplary embodiments, wireless local area network technologies, such as Wi-Fi or Wi-Fi HaLow, may be utilized to support higher data throughput and extended coverage within indoor environments. Wi-Fi HaLow, operating in sub-1 GHz frequency bands, may provide improved signal penetration through walls and reduced power consumption compared to conventional Wi-Fi, thereby supporting reliable transmission of real-time or near-real-time health-related data from the in-home health monitoring devices 260. These technologies may facilitate continuous clinical surveillance by enabling timely data transfer to the system 100 for risk analysis.

In some implementations, cellular communication technologies, including fourth-generation (4G) and fifth-generation (5G) cellular networks, may be employed to support mobility and wide-area connectivity for wearables 150 and in-home health monitoring devices 160. Cellular IoT connectivity enables data transmission during patient movement or outside a fixed residential environment, thereby supporting continuous clinical surveillance across diverse locations. In addition, location-based technologies, such as Global Positioning System (GPS), may be integrated with wearables 150 to provide mobility or activity context, which may be combined with physiological measurements to enhance assessment during continuous clinical surveillance. The described communication technologies may be used individually or in combination, and the selection of a particular technology may depend on factors such as power consumption, data rate, coverage requirements, and deployment environment.

In a further example, the system 100 may also include a mobile application that provides the patient with a personalized health feedback, educational health resources, and immediate access to clinician assistance upon displaying a red colored level risk alert in real time. Real time in the present context implies during collecting the biometric data and ongoing analysis of the risk level of diseases through specific AI modules. The mobile application could easily be installed in a smartphone or any other smart device implementing the system and can be easily accessed by the patient. The mobile application could remain active in the background of a mobile phone and share biometric data obtained by the wearable device. This way the patient can easily check out his or her overall health status and seek out timely help.

The system 100 may also trigger 140 visual risk alerts, based on the classified risk levels in the daily data trendline.

In an example, the visual risk alerts may be understood as ‘clear visual cues’ which may be displayed as one of risk scores, daily data trendline, and spotlight-style indicators, among others.

In another example, the displayed visual risk alerts may be indicated by at least three-colored levels, namely (A) green colored level indicating a stable patient condition, (B) yellow colored level indicating a moderate risk condition (cautionary) of the patient, and (C) red colored level indicating a high-risk condition of the patient, and the method may then include performing one or more additional steps based upon the alert. Thus, by translating large volumes of raw data into clear visual cues, the system enables faster, more targeted care decisions.

In an exemplary embodiment, the mobile interface for displaying the risk status may be tracked by the patient or family member or any other relevant person in the vicinity of the patient in case of an emergency. By timely tracking the risk status intervention can be done to prevent severity.

In another exemplary embodiment, the mobile interface for displaying the risk status may be tracked by a personal caregiver.

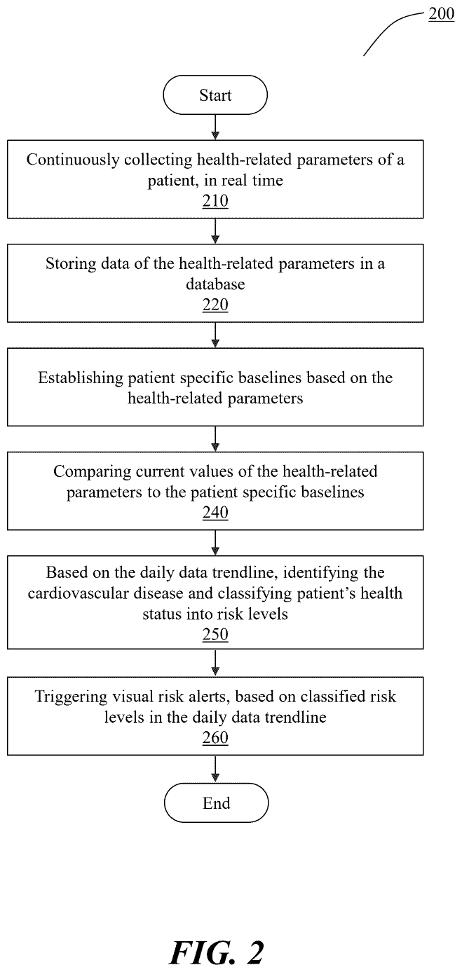

FIG. 2 illustrates a flowchart for continuous monitoring of cardiovascular diseases, in accordance with an embodiment of the present invention, in accordance with an embodiment of the present invention.

The method begins with step 210 which includes collecting health-related parameters of a patient, in real time, by a plurality of input devices.

At 220, the method may include storing data of the health-related parameters in a database.

At 230, the method may include establishing patient specific baselines based on the health-related parameters of the patient.

At 240, the method may include comparing current values of the health-related parameters to the patient specific baselines to generate a daily data trendline.

At 250, the method may include, identifying the cardiovascular disease and classifying the patient's health status into risk levels thereof, based on the daily data trendline.

At 260, the method may include triggering visual risk alerts, based on classified risk levels in the daily data trendline.

In an exemplary embodiment, the health-related parameters may include biometric parameters of the patient. The biometric parameters may be collected by wearables or clinical grade devices with connectivity to the server 110, where the biometric parameters may be at least one of heart rate (HR), heart rate variability (HRV), oxygen saturation (SpO2), step/activity data, and blood pressure of the patient, which may be collected on hourly or daily basis.

In an exemplary embodiment, the data may be collected on hourly or daily basis. A composite risk score may be generated by analyzing both absolute deviations and frequency of change of the health-related parameters from the collected data.

In an exemplary embodiment, the plurality of health-related parameters may be collected in one of every 15 minutes, 30 minutes, within a pre-defined time of five days. Fifteen-minute intervals capture subtle changes missed by standard once- or twice-daily checks.

In an exemplary embodiment, the method may include averaging hourly totals of the data of the health-related parameters to generate a daily risk score, after 24 hours. The daily risk score, provides a simple and unified metric for clinicians. Herein, the AI module smoothens out short-lived peaks or dips. Multi-day perspective helps distinguish short-term anomalies from actual risk progression.

In an exemplary embodiment, the method may include triggering the visual risk alert as:
Green (Stable): Patient's daily means remain within or near target ranges for the past five days.
Yellow (Caution): Moderate upward shift in systolic values observed over multiple days, indicating potential progression of hypertension.
Red (High Risk): Significant, sustained increase in daily means for multiple days, signaling imminent risk for secondary stroke.

In an exemplary embodiment, the method may include sending automated alerts via SMS, email, or EHR notifications to one of the patient and clinicians, via a dynamic interface 140 communicatively connected to the processing module, when the patient's health status transitions to an elevated risk level. The dynamic interface 140 may include a clinician web app 170 which provides an interactive dashboard with five-day trend graphs and color-coded alerts along with options for SMS/email notifications when a patient's status escalates. The clinician web app 170 may support messaging or telehealth modules for contacting patients flagged as yellow or red.

Also, the dynamic interface 140 may include a patient mobile app 180 which offers a simplified view of current status and daily/weekly progress. Also, the patient mobile app 180 provides educational tips or reminders triggered by yellow or red statuses to encourage self-care (e.g., medication adherence, lifestyle changes). Tus, dual app approach fosters collaborative care, empowering patients to take ownership of their health.

In an exemplary embodiment, the cardiovascular disease can be identified as at least one of congestive heart failure (CHF) decompensation, secondary stroke risk, and vascular health risk to the patient.

In an exemplary embodiment, to generate patient specific profiles in the database, the method may include collecting at least two weeks of baseline data, resting hearts rate (HR) ranges, typical oxygenation saturation (SpO₂) levels, and average daily steps.

In an exemplary embodiment, the method may include tracking hear rate Variability (HRV) and downward data trendline of SpO₂ levels for multi-days.

In an exemplary embodiment, the daily data trendline may comprise one of mild, moderate, and severe levels corresponding to early warning, caution, and high alert levels, respectively, based on both absolute values and deviations from the patient specific baseline. The below table 1 may be followed for the classification of the patient's health status:

The below table 1 may be followed for the classification of the patient's health status:

TABLE 1
Symptoms based classification of the patient's health status

| | Risk Alert | | |
|---|---|---|---|
| S. No. | level | Symptoms | Health-related parameter values |
| 1. | Early | Mild | HR ↑ of 5-10% from baseline |
| | Warning | variations | SpO₂ ↓ of 1-2 percentage points from |
| | | | baseline |
| | | | Steps ↓ of 5-10% for 2+ consecutive |
| | | | days |
| 2. | Caution | Moderate | HR ↑ >10% from baseline |
| | | variations | Persistent SpO₂ ↓ of >2 percentage |
| | | | points |
| | | | ≥10% reduction in steps for 3+ days |
| 3. | High Alert | Severe | HR ↑ >10-15% from baseline in a |
| | | variations | sustained pattern |
| | | | SpO₂ significantly below a threshold |
| | | | (e.g., <92%) or showing a rapid drop |
| | | | Steps ↓ of >20% over multiple days |

In an exemplary embodiment, the system 100 may aggregate the daily or hourly risk score into a green, yellow, and red visualization. The stoplight-style alerts offer simple and intuitive display for quick assessment of the patient's health status.

The below table 2 may be followed for the stoplight-style classification of the patient's health status.

TABLE 2
stoplight-style classification of the patient's health status

| | Risk Alert | | Health-related | |
|---|---|---|---|---|
| S. No. | level | Colour | parameter values | Action |
| 1. | Early | Green | HR within ±5% of | Routine monitoring |
| | Warning | | baseline | |
| | | | SpO2 near baseline | |
| | | | Steps/activity within ~5% | |
| | | | of normal | |
| 2. | Caution | Yellow | HR ↑ of 5-10% or | Alert care team; |
| | | | trending higher | consider telehealth |
| | | | SpO2 down 1-2 points | consult or medication |
| | | | from baseline | adjustment |
| | | | Steps ↓ by 5-10% for 2+ | |
| | | | days | |
| 3. | High Risk | Red | Sustained HR ↑ of >10% | Urgent evaluation, |
| | | | SpO2 significantly below | possible in-person |
| | | | acceptable range | intervention |
| | | | Major decline in activity | |
| | | | (>10-20% for multiple | |
| | | | days) | |

By collecting and analyzing continuous HR, SpO₂, and step count data, then translating these into an easy-to-read stoplight format, clinicians gain powerful, actionable insights into a patient's cardiovascular stability. By integrating machine learning, adaptive thresholds, and a stoplight-style risk interface, the clinicians can avail intuitive alerts to quickly identify early signs of CHF decompensation and intervene before full-blown hospitalizations occur.

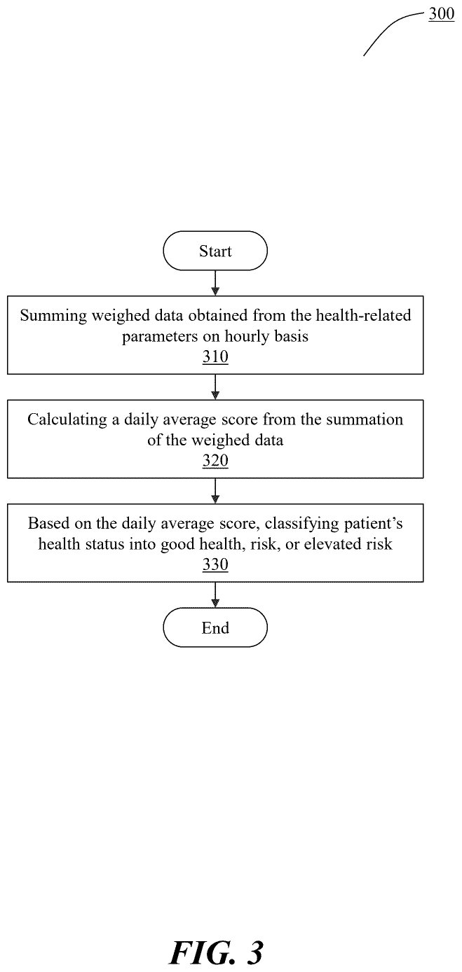

FIG. 3 illustrates a flowchart further illustrating the method from FIG. 2, in accordance with one or more embodiments of the present disclosure. At 310, the method may include summing weighed data obtained from the health-related parameters on an hourly basis. At 320, the method may include calculating a daily average score from the summation of the weighed data of the health-related parameters. At 330, the method may include, based on the daily average score, classifying the patient's health status into one of three tiers from one of good health, risk, or elevated risk. By assigning weights and drawing on multiple biometric inputs, such as heart rate, blood pressure, oxygen saturation, heart rate variability (HRV), and stress levels, a real-time or daily data trendline is generated to guide clinical decision-making and improve patient outcomes for patients at the risk of decreasing vascular health.

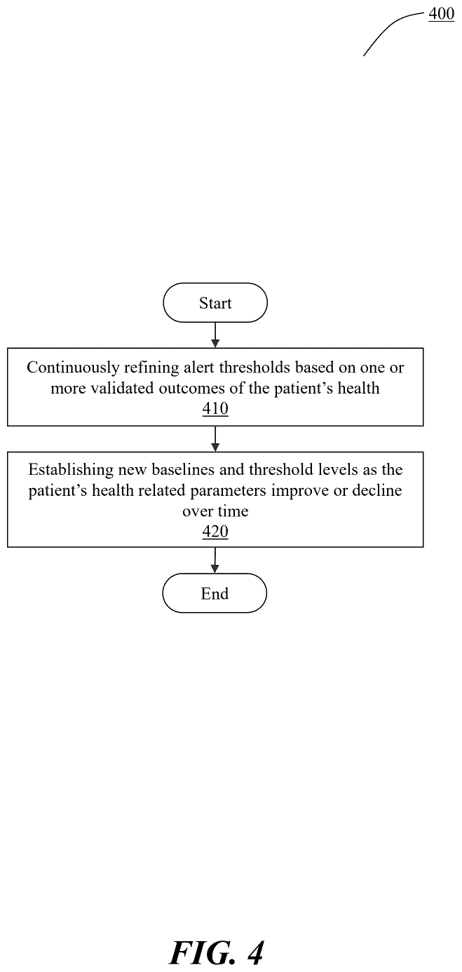

FIG. 4 illustrates a flowchart further illustrating the method from FIG. 2, in accordance with an embodiment of the present invention.

At 410, the method may include continuously refining alert thresholds based on one or more validated patient outcome.

At 420, the method may include establishing new baselines and threshold levels as the patient's health related parameters improve or decline over time. Thus, adaptive baselines and thresholds ensure each patient's evolving health status is accurately monitored.

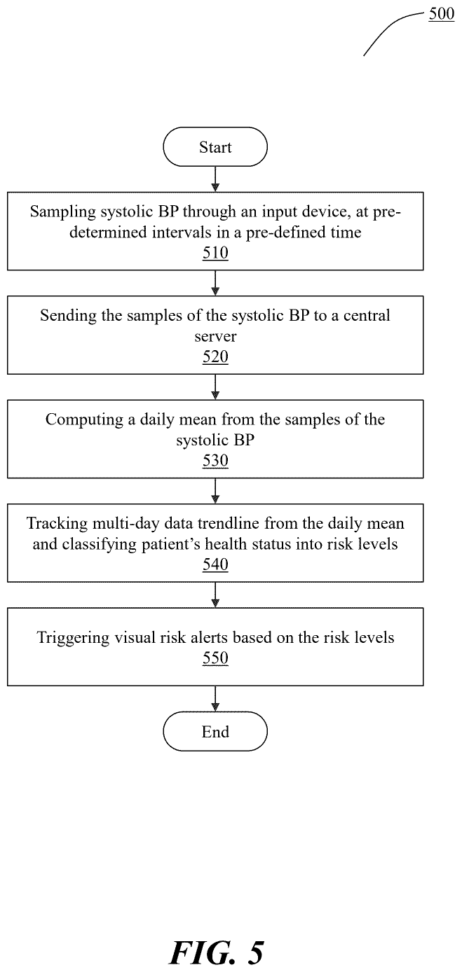

FIG. 5 illustrates a flowchart of a method for monitoring blood pressure to predict secondary stroke risk, in accordance with an embodiment of the present invention.

At 510, the method may include sampling systolic BP through an input device, at pre-determined intervals in a pre-defined time. Herein, by processing frequent (e.g., 15-minute) measurements, regressing them to a daily mean, and trending these means over a rolling five-day period, a color-coded risk indicator is provided through a web or mobile interface to clinicians and patients, respectively.

At 520, the method may include sending the samples of the systolic BP to a central server.

At 530, the method may include computing a daily mean from the samples of the systolic BP.

At 540, the method may include tracking multi-day data trendline from the daily mean and classifying patient's health status into risk levels.

At 550, the method may include triggering visual risk alerts based on the risk levels. The system may color-code the patient's health risk status as green (stable condition), yellow (cautionary condition), or red (high risk condition) and displays this information on both: (1) Clinician Web App: provides a comprehensive dashboard for medical professionals, showing multi-day trends and facilitating clinical decision-making, and (2) Patient Mobile App: offers a simplified interface so patients can track their own progress and engage in self-management.

In an implementation, the pre-determined intervals may be one of 15 minutes or 30 minutes, and the pre-defined time may be five days. In some embodiments, for sampling the systolic BP, the input device may be either a digit-based BP device or a standard BP cuff.

In an implementation, for tracking the multi-day data trendline, the daily means of the samples of the systolic BP may be analyzed for five-days via machine learning (ML) or statistical methods, such as linear regression and moving averages.

In an exemplary embodiment, various ML models may be utilized for analyzing the systolic BP in the five-day rolling window, for example, supervised models, unsupervised models, semi-supervised models, and reinforcement learning models. The supervised models use labeled data in which each data instance has a known category or value to which it belongs. This results in the supervised model to discover a relationship between input features and a target outcome. Examples of the supervised models may include support vector machines (SVM), random forest, K-nearest neighbors (KNN), and linear regression, etc. Further, the unsupervised models involve a difficult task of working with data which is not provided with pre-defined categories or label(s) and may be categorized into clustering, dimensionality reduction, and anomaly detection.

Further, deep neural networks (DNNs) may be used to perform complex computations because they contain many layers of these ML models or ML classifiers. At each layer, the DNN based models may create relationships between their inputs, thus enabling DNNs to extract more complex relationships and produce more sophisticated predictions or outputs. DNNs utilize large amounts of computational power and memory usage during the training and inference processes as the DNNs include millions of computational elements.

In an exemplary embodiment, the method may include excluding an oldest data point when each new day's mean enters a five-day window, thereby maintaining continuous trend oversight.

In an implementation, the method may include providing trend graphs and the visual risk indicators or automated alerts as one of an SMS, email, and electronic health record (EHR) notifications via a dynamic interface communicatively connected to the processing, when the patient's health status transitions to an elevated risk level.

In an implementation, the method may include bypassing the daily mean computation steps when a single systolic BP reading spikes above a critical threshold and sending urgent warnings to clinicians. The critical threshold may refer to a value above which a severe damage may occur to the patient's health, such as organ damage. Thus, urgent cases can be quickly referred to the clinicians for timely help and intervention.

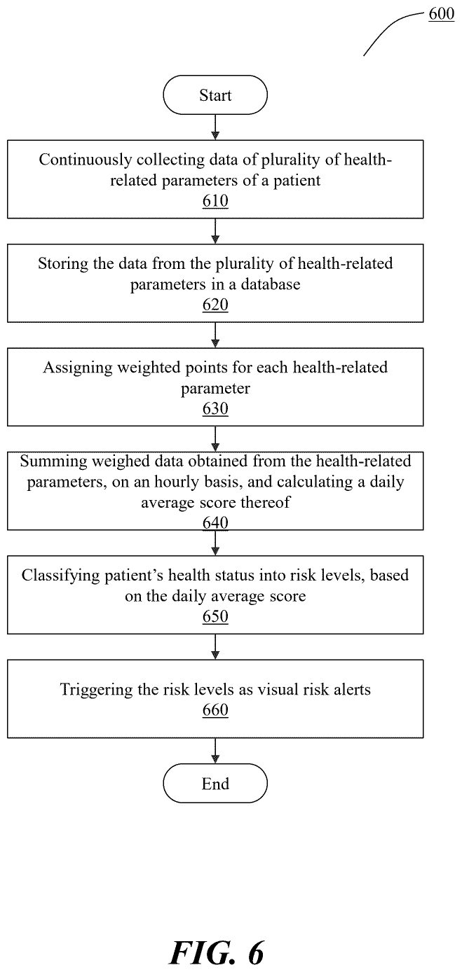

FIG. 6 illustrates a flowchart of a method 600 for indicating vascular health risk, in accordance with an embodiment of the present invention. At step 610, the method may include continuously collecting data of plurality of health-related parameters of a patient, from a plurality of input devices.

At step 620, the method may include storing the data from the plurality of health-related parameters in a database.

Further, at step 630, the method may recite assigning weighted points for each health-related parameter. Thereafter at step 640, the method may include summing weighed data obtained from the health-related parameters, on an hourly basis, and calculating a daily average score thereof.

In an exemplary embodiment, the method may include assigning weighted point values to individual health-related parameters based on their relative contribution to a composite risk score. Parameters exhibiting higher predictive relevance to a target physiological state are assigned greater weighted influence within the composite risk score.

In the disclosed AI-powered monitoring platform which is a multi-parameter risk monitoring platform, weighted point assignment enables effective fusion of heterogeneous health-related parameter data streams, including cardiovascular, oxygen saturation, and activity-related measurements. This weighted aggregation enables the processor to generate outputs that reflect combined physiological context rather than isolated measurements, earlier detection of meaningful physiological trends, improves sensitivity and specificity of risk levels, and reduces false positive events, thereby improving patient engagement.

For example, in a cardiovascular monitoring, heart rate variability (HRV) may be assigned a higher weighted point value relative to instantaneous heart rate measurements. While transient heart rate elevations may occur due to non-clinical factors, such as physical activity or stress, changes in HRV over time more reliably correlate with autonomic nervous system function. By assigning higher weighted points to HRV, noise-induced alerts may be the reduced and physiologically significant trends may be prioritized, resulting in more accurate risk stratification and timely-triggered interventions. These improvements collectively contribute to effective monitoring and more timely engagement with healthcare workflows, without requiring continuous clinician oversight.

At step 650, the method may include classifying patient's health status into risk levels, based on the daily average score. At the end step 660, the method may include triggering the risk levels as visual risk alerts. The categorization may be as:
Healthy: Score ≤10
Risk: Score >10 and ≤25
Elevated Risk: Score >25

In an exemplary embodiment, the method may include ingesting data from a plurality of input devices from one of smart rings, smartwatches, blood pressure cuffs, and other combinations of wearable devices, therefore the present method is device agnostic.

In an exemplary embodiment, the method may include averaging the hourly totals, after every 24 hours, to generate a daily average score.

In an exemplary embodiment, the patient's health status may be classified into risk levels as good health, risk, or elevated risk, upon detecting the patient's health status trending into the elevated risk level. For biometric inputs & scoring, the below values (table 3) of the biometric parameters may be considered as:

TABLE 3
Biometric inputs & scoring

Heart Rate: Moderate = 3 points; Severe = 10 points
Blood Pressure: Moderate = 5 points; Severe = 10 points
SpO₂: Moderate = 5 points; Severe = 10 points
Heart Rate Variability (HRV): Moderate = 3 points; Severe = 6 points
Stress: Moderate = 2 points; Severe = 4 points

After summing these parameters hourly, the system averages the total at day's end.

For daily risk score, the visual risk alerts may be classified as represented into table 4:

TABLE 4
Daily risk score

Healthy: 10 or below
Risk: above 10 but 25 or below
Elevated Risk: above 25

By mapping biometrics to these tiers, clinicians gain a user-friendly assessment tool that helps catch early warning signs of health deterioration.

In an exemplary embodiment, the method may include examining historical data of the patient, with a history of congestive heart failure (CHF) or comorbidities, for patterns or anomalies for analyzing impending health status, thereby enabling proactive care.

In an exemplary embodiment, the method may include implementing feedback steps from patient outcomes with one of successful interventions by clinicians and reduced hospital readmissions. In this manner, the AI-powered monitoring platform learns from patient outcomes, successful interventions, hospital readmissions, etc. to refine its predictive models over time.

In another exemplary embodiment, the disclosed system architecture may include a feedback loop. A clinician's input (validating or correcting flagged events) and responses from the users or patients (e.g., confirmation of symptoms) may be fed back into the AI module's training datasets. Over time, the system may refine its predictive accuracy, improving detection of truly significant variations in the physiological parameters.

Post-deployment, the methods 200, 300, 400, 500, and 600 may be optimized with mechanisms for continuous learning. Patient data may be anonymized and analyzed to identify improvement areas, and periodic updates may be applied to refine the capabilities of disclosed system.

In an exemplary embodiment, the method may include triggering immediate notifications as SMS, email, or alerts to clinicians, via a dynamic interface communicatively connected to the processor, when patient's health status transitions to an elevated risk level. If a patient is trending into “Elevated Risk,” immediate notifications are triggered (e.g., text, email, dashboard alerts) to care teams, prompting them to intervene.

In another exemplary embodiment, the disclosed system architecture may employ machine learning. In an example, the AI modules may be trained to learn each individual's normal baselines and adjust thresholds to minimize false alarms (e.g., naturally low HR for athletes or slightly elevated baseline BP for certain demographics). In another example, the disclosed system architecture may aggregate population-level patterns for more robust risk prediction.

FIG. 7 illustrates an exemplary view of an interface 700 depicting AI-driven health risk indicators, in accordance with an embodiment of the present invention. The interface 700 represents a web or mobile application and is custom-built for an end user, i.e., the clinician in the present case. The dashboard 700 may be accessed by the clinician through the web browser on a computer device or he may download an application store and install on his device. The interface 700 may be displayed on a clinician device which may be a touchscreen device, for example, a smartphone, tablet, or other computing device.

The interface 700 may include a central area that occupies a display screen of the clinician device. The central area of the interface 700 may indicate brief patient details 710, such as name of the patient, age, gender, and location. Further, the central area is designed for displaying stoplight statuses (green, yellow, and red) 720 or alert statuses 730, short historical trend graphs 740, and the other parameters related data 750 (ex, patient responses to mood related questions). Also, the central area highlights the CS 760. The central area of the dashboard 700 integrates near real-time updates under the automated remote continuous clinical surveillance to help the clinicians rapidly identify critical changes. In addition, the displaying stoplight statuses (green, yellow, and red) 720 provides a quick visual indicator for taking action in threatening situations.

In an exemplary embodiment, the risk scores may be indicated at least in a color-coded, text-based format, and graph-based format. By way of presentation of critical health related information in color and picture formats different ways, the clinician may easily differentiate from a normal situation to emergency situation and move on to the next patient if one case is resolved just by checking the risk status through the risk scores.

Moreover, the alert status may directly be shown with red color indicator in the interface 700 or an audio may be integrated for better triggering.

In an exemplary embodiment, a plus icon 770 may be displayed to the top left of the display screen. The plus icon 770 may provide plurality of options to the clinician. In an example, the plus icon 770 may enable the clinician to activate a voice-based input functionality, allowing for hands-free operation and voice commands. In another example, the plus icon 770 may include a settings tab for enabling customization options, such as language, voice command or video input, etc.

In addition, the self-report area of the display screen may display arrow signs icon for accessing previous data.

The disclosed method is capable of being implemented with multiple wearables which could be worn by the patient and are also connected to the server for biometric data monitoring.

In certain implementations, such monitoring may reduce the frequency of may reduce hospital readmissions while enabling recovery to occur in the home environment. Therefore, proactive detection and treatment may lower healthcare costs and enhance patient quality of life.

The stoplight system simplifies complex data streams, enabling rapid decision-making without data overload.

By alerting the clinicians and the patients to escalating hypertension early, the present invention can substantially reduce secondary stroke risks, enables proactive interventions, and improve long-term patient outcomes.

With the use of wearables and the disclosed implementation, both professional healthcare workflows (EHR integration, real-time alerts) and personal wellness tracking are packed in one system. This facilitates scaling and serviceability with minimal retraining, efforts, and cost.

In addition, by assigning weighed point values to distinct risk thresholds across multiple parameters, the system produces a holistic vascular health score. This approach enables earlier intervention, helping avoid costly exacerbations and readmissions. As the patients become more aware of how daily habits influence their risk scores, this potentially improves adherence to treatment.

The present invention provides a secure, intuitive Interface which displays real-time risk scores, alerts, and charts of key vitals. This way, care teams can drill down to see hourly metrics or long-term trends with near time tracking of patient's health status.

The present invention is capable of managing large patient populations, from a single practice to multi-facility health networks.

The discussed monitoring methods are perfect for CHF patients and stroke patients, but also adaptable for other cardiac conditions or comorbidities (e.g., COPD, diabetes).

By combining multiple biometric signals into a single score, the disclosed system simplifies clinical decision-making. Also, automated alerts eliminate the need for constant manual review, freeing clinicians to focus on higher-level care.

When discussing the invention, it is important to recognize that various techniques and steps are disclosed, each offering distinct advantages and capable of being employed independently or in combination with one another. Therefore, this description avoids redundant enumeration of all possible combinations of individual steps to maintain clarity. However, it should be noted that such combinations are fully encompassed within the scope of the invention and the accompanying claims. Consequently, the specification and claims should be interpreted with the understanding that these combinations are permissible and fall within the ambit of the invention.

In a case that no conflict occurs, the embodiments in the present disclosure and the features in the embodiments may be mutually combined. The foregoing descriptions are merely specific implementations of the present disclosure, but are not intended to limit the protection scope of the present disclosure. Any variation or replacement readily figured out by a person skilled in the art within the technical scope disclosed in the present disclosure shall fall within the protection scope of the present disclosure. Therefore, the protection scope of the present disclosure shall be subject to the protection scope of the claims.

The foregoing descriptions of specific embodiments of the present technology have been presented for purposes of illustration and description. They are not intended to be exhaustive or to limit the present technology to the precise forms disclosed, and obviously many modifications and variations are possible in light of the above teaching. The embodiments were chosen and described in order to best explain the principles of the present technology and its practical application, to thereby enable others skilled in the art to best utilize the present technology and various embodiments with various modifications as are suited to the particular use contemplated.

The invention claimed is:

1. A system for continuous disease risk monitoring, the system comprising:
an input device to continuously collect health-related parameters of a patient in real time;
a cloud-based server to store data of the health-related parameters in a database;
a processing module to:
establish patient specific baselines for the health-related parameters of the patient;
compare current values of the health-related parameters to the patient specific baselines to generate a daily data trendline;
based on the daily data trendline, identify at least one of congestive heart failure (CHF) decompensation risk, secondary stroke risk, and vascular health risk and classify patient's health status into risk levels thereof; and
trigger visual risk alerts, based on the classified risk levels in the daily data trendline; and
a dynamic interface communicatively connected to the processing module to send the visual risk alerts to the patient and clinicians.

2. The system of claim 1, wherein the input device is one of a wearable device, digit-based BP device or standard BP cuff, patch, and connected electrocardiogram (ECG) band or patch, and equivalents thereof.

3. The system of claim 1, wherein the processing module comprises at least one of an AI-based module or a ML based module.

4. A method for continuous monitoring of cardiovascular diseases, implemented by a processing module, the method comprising:
continuously collecting health-related parameters of a patient, in real time, by a plurality of input devices;
storing data of the health-related parameters in a database;
establishing patient specific baselines based on the health-related parameters of the patient;
comparing current values of the health-related parameters to the patient specific baselines to generate a daily data trendline;
based on the daily data trendline, identifying risk of a cardiovascular disease and classifying patient's health status into risk levels thereof; and
triggering visual risk alerts, based on classified risk levels in the daily data trendline.

5. The method of claim 4, wherein the health-related parameters comprise biometric parameters of the patient, and wherein the biometric parameters are at least one of heart rate (HR), heart rate variability (HRV), oxygen saturation (SpO₂), step/activity data, and blood pressure of the patient, which are collected on hourly or daily basis.

6. The method of claim 4, wherein the data is collected on hourly or daily basis, and wherein a composite risk score is generated by analyzing both absolute deviations and frequency of change of the health-related parameters from the collected data.

7. The method of claim 4, wherein the plurality of health-related parameters is collected in one of every 15 minutes, 30 minutes, within a pre-defined time of five days.

8. The method of claim 4, wherein the method comprises:
summing weighed data obtained from the health-related parameters on an hourly basis;
calculating a daily average score from the summation of the weighed data of the health-related parameters; and
based on the daily average score, classifying patient's health status into one of three tiers from one of good health, risk, or elevated risk.

9. The method of claim 4, wherein the method comprises averaging hourly totals of the data of the health-related parameters to generate a daily risk score, after 24 hours.

10. The method of claim 4, wherein the method comprises:
continuously refining alert thresholds based on one or more validated patient outcomes of the patient's health status, and
establishing new baselines and threshold levels as the patient's health related parameters improve or decline over time.

11. The method of claim 4, wherein the method comprises sending automated alerts via SMS, email, or EHR notifications to one of the patient and clinicians, via a dynamic interface communicatively connected to the processing module, when patient's health status transitions to an elevated risk level.

12. The method of claim 4, wherein the cardiovascular disease can be identified as at least one of congestive heart failure (CHF) decompensation, secondary stroke risk, and vascular health risk to the patient.

13. The method of claim 4, wherein to generate patient specific profiles in the database, the method comprises collecting at least two weeks of baseline data, resting hearts rate (HR) ranges, typical oxygenation saturation (SpO₂) levels, and average daily steps.

14. The method of claim 4, wherein the method comprises tracking hear rate Variability (HRV) and downward data trendline of SpO₂ levels for multi-days.

15. The method of claim 4, wherein the daily data trendline comprise one of mild, moderate, and severe levels corresponding to early warning, caution, and high alert levels, respectively, based on both absolute values and deviations from the patient specific baseline.

16. A method for monitoring blood pressure (BP) to predict secondary stroke risk implemented by a processor in an AI module, the method comprising:
sampling systolic BP through an input device, at pre-determined intervals in a pre-defined time;
sending the samples of the systolic BP to a central server;
computing a daily mean from the samples of the systolic BP;
tracking multi-day data trendline from the daily mean and classifying patient's health status into risk levels; and
triggering visual risk alerts based on the risk levels.

17. The method of claim 16, wherein the pre-determined intervals is one of 15 minutes or 30 minutes, and the pre-defined time is five days.

18. The method of claim 16, wherein for sampling the systolic BP, the input device is either a digit-based BP device or a standard BP cuff.

19. The method of claim 16, wherein for tracking the multi-day data trendline, the daily means of the samples of the systolic BP are analyzed for five-days via machine learning (ML) or statistical methods.

20. The method of claim 16, wherein the method comprises excluding an oldest data point when each new day's mean enters a five-day window.
